(19) United States
(12) Patent Application Publication
(10) Pub. No.: US 2007/0191638 A1
(43) Pub. Date: Aug. 16, 2007

(54) Processes for the synthesis of N,N'-substituted 1,3-diketimines

(76) Inventor: Kyung-Ho Park, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/351,151

(22) Filed: Feb. 10, 2006

(51) Int. Cl. C07C 249/02

(52) U.S. Cl. 564/272; 564/278

(57) ABSTRACT

Processes for the synthesis of N,N′-substituted 1,3-diketimines (III) from the reaction of aliphatic ketimines with iminothioethers in the presence of base are provided. The processes are particularly useful for making aliphatic N,N′-unsymmetrically substituted 1,3-diketimines.

### FIELD OF THE INVENTION

This invention provides processes for the synthesis of N,N′-substituted 1,3-diketimines.

### TECHNICAL BACKGROUND

Copper complexes are of interest as precursors for the preparation of thin copper films. Creation of such metallic films, for example by chemical vapor deposition or atomic layer deposition, could be used in the manufacture of a wide variety of electronic devices.

Hexafluoroacetylacetonato(trimethylsilylethylene)copper(I), (D. Bollmann, R. Merkel, and A. Klumpp Microelectronic Eng. 1997, 37/38, 105, and reference there-in) has been widely tested for this application, but the presence of oxygen and fluorine in this precursor may be detrimental to the desired performance, including device efficiency (P. Motte, M. Proust, J. Rorres, Y. Gobil, Y. Morand, J. Palleau, R. Pantel, M. Juhel Microelectronic Eng. 2000, 50, 369). Volatile, oxygen- and halogen-free complexes of copper are desired.

Alternative ligands, such as 1,3-diketimines, have also been investigated as metal complex precursors for microchip interconnect layers. Preparation of symmetrically substituted 1,3-diketimines and their homoleptic metal complexes of the form ML₂ have been described by S. G. McGeachin (Canadian Journal of Chemistry, 1968, 46, 1903-1912).

U.S. Pat. No. 6,939,578 discloses methods for preparing copper complexes derived from both N,N′-symmetrical and N,N′-unsymmetrical 1,3-diimine ligands. The N,N′-unsymmetrically substituted 1,3-diimines are expected to be more volatile than their symmetrically substituted counterparts, due to the less compacted mode of molecular stacking originating from the unsymmetrical ligand.

DE 2,707,658 and U.S. Pat. No. 4,130,652 describe the preparation of monocyclic 1,3-diketimines having aromatic substituents in the presence of acid.

K-. H. Park (J. of Organic Chemistry, 2005, 70, 2075-2081) discloses the preparation of N,N′-substituted 1,3-diketimines in the presence of base.

### SUMMARY OF THE INVENTION

One embodiment of this invention provides a process for the synthesis of N,N′-substituted 1,3-diketimines (III) from the reaction of aliphatic ketimines (I) with iminothioethers (II) in the presence of base.

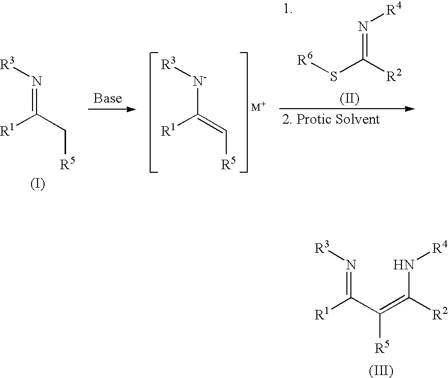

One embodiment of this invention is a process comprising:

a. reacting R³N═C(R¹)CH₂R⁵ with an alkali metal or alkaline earth metal base in a polar aprotic solvent to form a metalloenamine, [R³NC(R¹)CHR⁵]⁻M⁺, where M is an alkali metal or an alkaline earth metal;

b. reacting the metalloenamine with R⁶SC(R²)═NR⁴ to form a 1,3-diketiminate salt; and

c. treating the diketiminate salt with a protic solvent to form a 1,3-diketimine, R³N═C(R¹)C (R⁵)═C(R²)NHR⁴,

wherein

R¹ is selected from the group consisting of C₁-C₅ linear alkyl groups and C₆-C₁₂ aryl groups; and

R³ and R⁵ are independently selected from the group consisting of hydrogen, C₁-C₅ linear alkyl groups and C₆-C₁₂ aryl groups; or

(R¹, R³) or (R¹, R⁵) taken together are (CR⁷R⁸)ₘ, where R⁷ and R⁸ are independently selected from the group consisting of hydrogen and C₁-C₅ alkyl, and m is 3, 4 or 5;

R² and R⁴ are independently selected from the group consisting of hydrogen and C₁-C₅ alkyl groups and C₆-C₁₂ aryl groups; or

(R², R⁴) taken together are (CR⁹R¹⁰)ₙ, where R⁹ and R¹⁰ are independently selected from the group consisting of hydrogen and C₁-C₅ alkyl, and n is 3, 4 or 5; and

R⁶ is selected from a group consisting of C₁-C₁₀ alkyl and C₆-C₁₀ aryl groups.

### DETAILED DESCRIPTION

Applicant has discovered an efficient synthesis of N,N′-substituted 1,3-diketimines that may be used to make metal precursors with sufficient volatility to be useful in CVD or ALD processes for the deposition of thin metal films. This process is especially useful for making aliphatic N,N′-unsymmetrically substituted 1,3-diketimines.

In one embodiment of this invention, the desired 1,3-diketimines (III) are obtained substantially pure from the reaction of ketimines (I) with iminothioethers (II) in the presence of base.

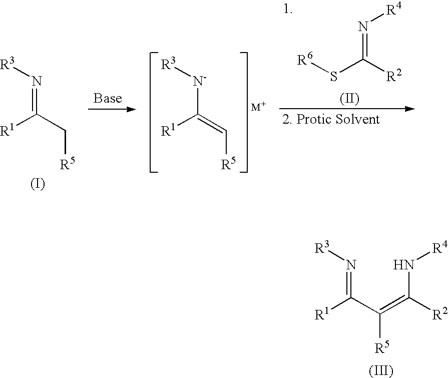

One embodiment of this invention is a process comprising:

a. reacting R³N═C(R¹)CH₂R⁵ with an alkali metal or alkaline earth metal base in a polar aprotic solvent to form a metalloenamine, [R³NC(R¹)CHR5]⁻M⁺, where M is an alkali metal or an alkaline earth metal;

b. reacting the metalloenamine with R⁶SC(R²)═NR⁴ to form a 1,3-diketiminate salt; and

c. treating the diketiminate salt with a protic solvent to form a 1,3-diketimine, R³N═C(R¹)C (R⁵)═C(R²)NHR⁴,

wherein

R¹ is selected from the group consisting of C₁-C₅ linear alkyl groups and C₆-C₁₂ aryl groups; and

R³ and R⁵ are independently selected from the group consisting of hydrogen, C₁-C₅ linear alkyl groups and C₆-C₁₂ aryl groups; or

(R¹, R³) or (R¹, R⁵) taken together are (CR⁷R⁸)ₘ, where R⁷ and R⁸ are independently selected from the group consisting of hydrogen and C₁-C₅ alkyl, and m is 3, 4 or 5;

R² and R⁴ are independently selected from the group consisting of hydrogen and C₁-C₅ alkyl groups and C₆-C₁₂ aryl groups; or

(R², R⁴) taken together are (CR⁹R¹⁰)ₙ, where R⁹ and R¹⁰ are independently selected from the group consisting of hydrogen and C₁-C₅ alkyl, and n is 3, 4 or 5; and

R⁶ is selected from a group consisting of C₁-C₁₀ alkyl and C₆-C₁₀ aryl groups.

If any of the R groups, R¹-R¹⁰, is an alkyl or aryl group, it can be either substituted or unsubstituted. Suitable substitutents include alkylsilyl groups, arylsilyl groups, ether groups, alkyl groups, aryl groups, haloaryl groups and CF₃-substituted aryl groups.

In the process of this invention, an aliphatic ketimine (I) is deprotonated by a base in a polar, aprotic solvent. Suitable bases include alkali or alkaline earth metal hydrides such as NaH or CaH₂, lithium alkylamides such as lithium diisopropylamide, lithium hexamethyidisilazane, sodium hexamethyldisilazane, alkyl lithiated bases such as butyl lithium, aryl lithiated bases such as phenyl lithium, and alkylmagnesium halides such as methylmagnesium bromide.

Suitable polar aprotic solvents for the deprotonation reaction include tetrahydrofuran, ether, dimethoxyethane, dioxane, and diglyme.

The metalloenamine from ketimine (I) reacts with the electrophile iminothioether (II), providing a 1,3-diketiminate metal salt, which is protonated by protic solvent. Suitable solvents for the protonation of 1,3-diketiminate salt include, but are not limited to water, methanol, ethanol, and propanol.

In one embodiment of this invention, ketimine (I) is added dropwise to a mixture of the sodium or lithium base in the aprotic solvent at −78° C. to 0° C. under an inert atmosphere. After stirring the mixture at this temperature for 0.5 hr to 2 hr, iminoether (II) is added dropwise at at −78° C. to 0° C. under an inert atmosphere. The temperature is allowed to increase to room temperature over a period of 2-6 hours and the resultant mixture is stirred for 2 days. The reaction mixture is concentrated under reduced pressure, and then protic solvent is slowly added to the residue. After removing the solvent under reduced pressure, a nonpolar hydrocarbon solvent such as pentane or hexane is added to the residue. The mixture is filtered, then after concentration of the filtrate under reduced pressure, the product is isolated by vacuum distillation.

In other embodiments of this invention, the solution of base is added dropwise to ketimine (I). Similarly, the solution of the metalloenamine, [R³NC(R¹)CHR⁵]⁻M⁺, can be added dropwise to the iminoether (II).

Ketimines useful in the process of this invention can be synthesized by the reaction of ketone derivatives with amines. For example, acetone and isobutylamine are mixed together in the presence of acid catalyst such as hydrochloric acid to provide the ketimine (I), N-isopropylideneisobutylamine, as described by W. H. Bunnelle (Synthesis 439, (1997)).

Similarly, iminothioethers can be synthesized by the alkylation of thioamide derivatives with alkylating agents such as iodomethane or Meerwein's salt, as described in M. A. Casadei (Synthetic Communication, 1983, 20, 753-759).

### EXAMPLES

Unless otherwise stated, all organic reagents are available from Sigma-Aldrich Corporation (Milwaukee, Wis., USA). The ketimines (I) and iminothioethers (II) were prepared by methods described by W. H. Bunnelle ibid. and M. A. Casadei ibid., respectively.

### Example 1

### Preparation of

### ((1Z, 3E)-4-Aza-1,3,6-trimethylhepta-1,3-dienyl)methylamine

To a solution of diisopropylamine (10.29 g, 101.8 mmol, 2.1 eq) in THF (200 mL) was added n-BuLi (35.2 mL, 101.8 mmol, 2.1 eq, 2.89 M in hexane) dropwise at −78° C. The deprotonation mixture was stirred at −78° C. for 30 min, then stirred at −10° C. for another 30 min. Then, a solution of N-isopropylideneisobutylamine, I (wherein R¹=Me, R³=isobutyl, R⁵=hydrogen), (7.13 g, 63 mmol, 1.3 eq) in THF (20 mL) was added dropwise to the deprotonation mixture at −10° C. After stirring the resulting mixture for 40 min at −10° C., methyl N-methylthioacetimidate, (5 g, 48.45 mmol) solution in THF (15 mL) was added to the mixture dropwise at −10° C. The resultant mixture was stirred overnight as the temperature was allowed to gradually rise to room temperature. The reaction mixture was concentrated under reduced pressure, then MeOH (30 mL) was slowly added to the residue. After removing the solvent under reduced pressure, pentane (100 mL) was added to the residue. The mixture was filtered, then the filtrate was concentrated under reduced pressure, followed by vacuum distillation (35° C., 72 mtorr) to afford desired product (4.3 g, 53%) as a colorless oil. ¹H NMR (500 MHz, C₆D₆) δ 11.41 (s, br, 1H), 4.62 (s, 1H), 2.94 (d, 2H, J=6.6 Hz), 2.82 (s, 3H), 1.77 (m,1H), 1.71 (s, 3H), 1.66 (s, 3H), 0.94 (d, 6H, J=6.4 Hz); ¹³C NMR (125 MHz, C₆D₆) δ 161.9, 159.8, 95.1, 54.6, 33.4, 30.4, 20.7, 19.4, 18.9.

### Example 2

### Preparation of

### ((1Z)-1-Methyl-2-(1-pyrrolin-2-yl)vinyl)(2-methylpropyl)amine

To the solution of diisopropylamine (10.29g, 101.8 mmol, 2.1 eq) in THF (200 mL) was added n-BuLi (46.3 mL, 101.8 mmol, 2.1 eq, 2.2 M in hexane) dropwise at −78° C. The mixture was stirred at −78° C. for 30 min, then stirred at −10° C. for another 30 min. Then, N-isopropylideneisobutylamine (7.13g, 63 mmol, 1.3 eq) solution in THF (20 mL) was added dropwise to the mixture at −10° C. After stirring the mixture for 40 min at the same temperature, 2-methylthio-1-pyrroline (5.58 g, 48.45 mmol) solution in THF (15 mL) was added to the mixture dropwise at −10° C. The resultant mixture was stirred overnight as the temperature was allowed to gradually rise to room temperature. The reaction mixture was concentrated under reduced pressure, then MeOH (20 mL) was slowly added to the residue. After removing the solvent under reduced pressure, pentane (120 mL) was added to the residue. The mixture was filtered, then the filtrate was concentrated under reduced pressure, followed by vacuum distillation (54° C., 102 mtorr) to afford desired product (5.8 g, 66%) as a colorless oil. ¹H NMR (500 MHz, CDCl₃) δ 9.92 (s, br, 1H), 4.54 (s, 1H), 3.82 (t, 2H, J=7.1 Hz), 2.98 (d, 2H, J=6.7 Hz), 2.46 (t, 2H, J=8.0 Hz), 1.90 (s, 3H), 1.77-1.69 (m, 3H), 0.92 (d, 6H, J=7.3 Hz); ¹³C NMR (125 MHz, CDCl₃) δ 173.9, 154.4, 87.3, 60.1, 51.0, 37.7, 29.6, 22.4, 20.0, 19.1.

### Example 3

### Preparation of

### 2-(Pyrrolidin-2-ylidenemethyl)-3,4,5,6-tetrahydropyridine

To the solution of diisopropylamine (24 g, 237 mmol, 2.1 eq) in THF (400 mL) was added n-BuLi (108 mL, 237 mmol, 2.1 eq, 2.2 M in hexane) dropwise at −78° C. The mixture was stirred at −78° C. for 30 min, then stirred at −10° C. for another 30 min. Then, 2-methyl-3,4,5,6-tetrahydropyridine (14.3 g, 147 mmol, 1.3 eq) solution in THF (20 mL) was added dropwise to the mixture at −10° C. After stirring the mixture for 40 min at the same temperature, 2-methylthio-1-pyrroline (13 g, 112.8 mmol) solution in THF (20 mL) was added to the mixture dropwise at −10° C. The resultant mixture was stirred overnight as the temperature was allowed to gradually rise to room temperature. The reaction mixture was concentrated under reduced pressure, then MeOH (100 mL) was slowly added to the residue. After removing the solvent under reduced pressure, pentane (200 mL) was added to the residue. The mixture was filtered, then the filtrate was concentrated under reduced pressure, followed by vacuum distillation (58° C., 46 mtorr) to afford desired product (16 g, 86%) as an oil. ¹H NMR (500 MHz, CD₂Cl₂) δ 9.08 (s, br, 1H), 4.49 (s, 1H), 3.78 (t, 2H, J=7.1 Hz), 3.27 (t, 2H, J=6.0 Hz), 2.46 (t, 2H, J=7.9 Hz), 2.34 (t, 2H, J=6.7 Hz), 1.73 (m, 2H), 1.67 (m, 2H); ¹³C NMR (125 MHz, CD₂Cl₂) δ 173.9, 156.1, 87.0, 60.2, 42.0, 38.0, 29.6, 23.9, 22.8, 21.5.

### Example 4

### Preparation of 2-(Pyrrolidin-2-ylidenemethyl)-1-Pyrroline

To a solution of diisopropylamine (11.1 g, 109.7 mmol) in THF (200 mL) was dropwise added n-BuLi (2.89 M, 37.97 mL, 109.7 mmol) at −78° C. under nitrogen. Once all the n-BuLi was added, the temperature was adjusted to −5° C., and the reaction mixture was stirred for 30 min. Then a solution of 2-methyl-1-pyrroline (5.65 g, 67.9 mmol) in THF (15 mL) was added dropwise to the reaction mixture at −5° C., and then stirred. After 30 min, 2-methylthio-1-pyrroline (6.02 g, 52.3 mmol) was added dropwise over 30 min at −78° C. The reaction mixture was stirred as the temperature was allowed to gradually rise to room temperature, and was continuously stirred at room temperature overnight. THF solvent was removed under reduced pressure, then 50 mL of methanol was added dropwise to the residue. After removing all of the volatile solvent, pentane (2×100 mL) was added to the residue, and the mixture was filtered. Concentration of the filtrate under reduced pressure, followed by vacuum distillation (65° C. at 110 mtorr), delivered 6.2 g of product (79%). ¹H NMR (CD₂Cl₂, 500 MHz): δ 7.89 (s, br, 1H), 4.65 (s, 1H), 3.64 (t, 2H, J=7.2 Hz), 2.51 (t, 2H, J=8.0 Hz), 1.85 (m, 2H). ¹³C NMR (CD₂Cl₂, 125 MHz): δ 167.0, 81.7, 53.7, 34.8, 23.2.

I claim:

1. A process comprising:
a. reacting R³N═C(R¹)CH₂R⁵ with an alkali metal or alkaline earth metal base in a polar aprotic solvent to form a metalloenamine, [R³NC(R¹)CHR⁵]⁻M⁺, where M is an alkali metal or an alkaline earth metal;
b. reacting the metalloenamine with R⁶SC(R²)═NR⁴ to form a 1,3-diketiminate salt; and
c. treating the diketiminate salt with a protic solvent to form a 1,3-diketimine, R³N═C(R¹)C (R⁵)═C(R²)NHR⁴,
wherein
R¹ is selected from the group consisting of C₁-C₅ linear alkyl groups and C₆-C₁₂ aryl groups; and
R³ and R⁵ are independently selected from the group consisting of hydrogen, C₁-C₅ linear alkyl groups and C₆-C₁₂ aryl groups; or
(R¹, R³) or (R¹, R⁵) taken together are (CR⁷R⁸)ₘ, where R⁷ and R⁸ are independently selected from the group consisting of hydrogen and C₁-C₅ alkyl, and m is 3, 4 or 5;
R² and R⁴ are independently selected from the group consisting of hydrogen and C₁-C₅ alkyl groups and C₆-C₁₂ aryl groups; or
(R², R⁴) taken together are (CR⁹R¹⁰)ₙ, where R⁹ and R¹⁰ are independently selected from the group consisting of hydrogen and C₁-C₅ alkyl, and n is 3, 4 or 5; and
R⁶ is selected from a group consisting of C₁-C₁₀ alkyl and C₆-C₁₀ aryl groups.

2. The process of claim 1, wherein R¹ and R² are independently selected from the group consisting of C₁-C₅ alkyl; and R³, R⁴ and R⁵ are independently selected from the group consisting of hydrogen and C₁-C₅ alkyl.

3. The process of claim 1, wherein R¹ is independently selected from the group consisting of C₁-C₅ alkyl; R³ and R⁵ are independently selected from the group consisting of hydrogen and C₁-C₅ alkyl; and (R²,R⁴) are taken together as (CH₂)ₙ, where n is 3, 4 or 5.

4. The process of claim 1, wherein (R¹,R³) are taken together as (CH₂)ₘ, where m is 3, 4 or 5; (R²,R⁴) are taken together as (CH₂)ₙ, where n is 3, 4 or 5; and R⁵ is selected from the group consisting of hydrogen and C₁-C₅ alkyl.

5. The process of claim 1, wherein the base is selected from the group consisting of NaH, CaH₂, lithium alkylamides, lithium hexamethyldisilazane, sodium hexamethyldisilazane, alkyl lithiated bases, aryl lithiated bases, and alkylmagnesium halides.

6. The process of claim 5, wherein the alkyl lithiated base is butyl lithium, the aryl lithiated base is phenyl lithium and the lithium alkylamide is lithium diisopropylamide.

7. The process of claim 1, wherein the polar aprotic solvent is selected from the group consisting of tetrahydrofuran, ether, dioxane, and diglyme.

8. The process of claim 1, wherein the protic solvent is selected from the group of C₁-C₅ alcohols.
